(19) United States
(12) Patent Application Publication
(10) Pub. No.: US 2024/0124844 A1
(43) Pub. Date: Apr. 18, 2024

(54) METHOD FOR PREPARING COMPOSITION COMPRISING MESENCHYMAL STEM CELL, EXTRACELLULAR VESICLE PRODUCED BY MESENCHYMAL STEM CELL, AND GROWTH FACTOR, COMPOSITION PREPARED BY THE METHOD, AND USE OF COMPOSITION FOR TREATING ARTHRITIS

(71) Applicant: GWO XI STEM CELL APPLIED TECHNOLOGY CO., LTD., Hsinchu County (TW)

(72) Inventors: Chia-Hsin Lee, Hsinchu County (TW); Po-Cheng Lin, Hsinchu County (TW); Yong-Cheng Kao, Hsinchu County (TW); Ming-Hsi Chuang, Hsinchu County (TW); Chun-Hung Chen, Hsinchu County (TW); Chao-Liang Chang, Hsinchu County (TW); Kai-Ling Zhang, Hsinchu County (TW)

(21) Appl. No.: 18/376,459

(22) Filed: Oct. 4, 2023

Related U.S. Application Data
(60) Provisional application No. 63/414,920, filed on Oct. 11, 2022.

(30) Foreign Application Priority Data
Sep. 27, 2023 (TW) 112137021

(51) Int. Cl. C12N 5/0775; A61P 19/02

(52) U.S. Cl. CPC C12N 5/0667; A61P 19/02; C12N 2500/32; C12N 2500/38; C12N 2501/10

(57) ABSTRACT

The present disclosure provides a method for preparing a composition including mesenchymal stem cells, extracellular vesicles produced by the mesenchymal stem cells, and growth factors, the composition prepared by the method, and use of the composition for treating arthritis. The composition of the present disclosure achieves the effect of treating arthritis through various efficacy experiments.

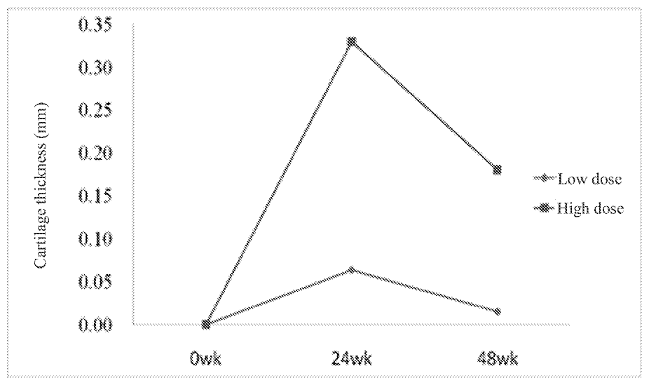

### CROSS-REFERENCE TO RELATED APPLICATION

This application claims priorities of Provisional application No. 63/414,920, filed on Oct. 11, 2022, and Taiwan patent application No. 112137021, filed on Sep. 27, 2023 the content of which is incorporated herein in its entirety by reference.

### BACKGROUND OF THE INVENTION

### 1. Field of the Invention

The present invention relates to a method for preparing a composition comprising mesenchymal stem cells, extracellular vesicles produced by the mesenchymal stem cells, and growth factors, the composition prepared by the method, and use of the composition for treating arthritis.

### 2. The Prior Art

Arthritis is one of the most common chronic diseases in the world. It is mainly caused by the deterioration of the cartilage of the joints or inflammation of the connective tissue, which causes joint pain and interferes with the normal movement of the joints. Common arthritis includes osteoarthritis (OA), rheumatoid arthritis, rheumatic arthritis, septic arthritis, traumatic osteoarthritis, autoimmune arthritis, ankylosing arthritis, etc. The symptoms and signs of arthritis mainly occur in the affected joints, usually the weight-bearing joints or interphalangeal joints of the fingers. They can occur in the knees, hip joints, ankles, hands, shoulder joints, back, and neck.

Osteoarthritis, also known as degenerative arthritis, is a symptom of diarthrosis failure. The main symptoms are degeneration of articular cartilage and friction between bones, resulting in joint pain, tenderness, stiffness, locking, joint effusion, decreased joint mobility, loss of joint space, osteophyte formation, cyst formation, joint deformation, and in severe cases may lead to disability.

The World Health Organization (WHO) designates October 12 every year as “World Arthritis Day”. The global prevalence of osteoarthritis increased from 247.51 million in 1990 to 527.81 million in 2019, an increase of 113.25%. According to Precision Reports, the global stem cell market for the treatment of osteoarthritis is expected to increase from US$22 million in 2021 to US$107.3 million in 2028, with a compound growth rate of 25.1% from 2022 to 2028. In Taiwan, according to the National Health Insurance Medical Statistics Annual Report released by the Statistics Office of the Ministry of Health and Welfare in 2021, there were 3,893,506 outpatient and inpatient visits for knee osteoarthritis in Taiwan, and the medical cost statistics were approximately NT$5.88 billion. It can be seen that osteoarthritis market potential is considerable. Current treatments for osteoarthritis include: arch pad correction, functional knee pads, rehabilitation medicine electrotherapy, glucosamine, chondroitin, non-steroidal anti-inflammatory drugs (NSAID), intra-articular injection of corticosteroids, intra-articular injection of hyaluronic acid (HA), injection of platelet rich plasma (PRP), autologous chondrocyte implantation (ACI), partial knee replacement, total knee replacement (TKR), and the like.

The age group of patients with osteoarthritis is declining. Originally, most patients were over 50 years old, but in recent years, patients in their 30s and 40s have begun to appear. It is no longer a disease unique to the elderly. In the United States, it is second only to heart disease as the “culprit” that makes people unable to work. In Taiwan, it is also the main cause of adult disability. Because patients with degenerative arthritis often experience physical pain, joint pain, or other systemic discomforts, they not only have difficulty sleeping, are easily tired, and may also lose weight. Severe joint injuries and deformations greatly affect mobility and reduce opportunities to go out. Some patients can even stay in bed for long periods of time and cannot go out at all, seriously affecting health-related quality of life (HRQOL).

Mesenchymal stem cells (MSCs) are regarded as a therapeutic drug because of their regenerative, immunomodulatory and paracrine properties. They provide new treatment models for patients with osteoarthritis. There are currently 17 types of mesenchymal stem cells from different sources, including adipose tissue, umbilical cord blood, amniotic membrane, amniotic fluid, Wharton's Jelly, placenta, synovium, dental pulp, bone marrow and peripheral blood, etc. Studies have shown that autologous stem cells can be used to treat osteoarthritis. However, autologous stem cell therapy requires removing tissue from the patient and then culturing the stem cells before treatment, which takes a long time. In addition, autologous stem cell therapy has limitations for patients who cannot provide their own tissues, such as the elderly, the infirm, those with infectious diseases, or those who cannot wait due to the course of the disease. Allogeneic stem cell therapy overcomes this limitation. Allogeneic stem cells can be used to treat osteoarthritis by directly injecting stem cells into the joint cavity of the affected area. After completing the treatment, you can leave the hospital. There is no need to wait for cell culture amplification time, no need for hospitalization, no need for surgery and saving treatment steps, which increases the willingness of patients to use it, thus increasing the popularity and convenience of the product. Vega and other scholars have confirmed that allogeneic bone marrow mesenchymal stem cells (BMMSCs) are used to treat osteoarthritis and can reduce the discomfort caused by osteoarthritis, but have no significant effect on improving the quality of life. Another article has similar results to the study by Vega et al. It uses allogeneic adipose-derived MSCs (ADSCs) to treat osteoarthritis and can also reduce the discomfort caused by osteoarthritis. However, the results of the quality of life scale are reduced. Current research on allogeneic stem cell treatment for osteoarthritis indicates that it can only reduce arthritis discomfort by about 10% one week after treatment, and it takes 12 weeks to reduce arthritis discomfort by 56%. In summary, there is a need in the osteoarthritis treatment market for products that can quickly and effectively improve quality of life.

At present, clinical drug treatments for arthritis have limited effect and have serious side effects, and many patients cannot continue to treat. More importantly, the drug only alleviates the symptoms, but fails to fundamentally solve the problem, so how to develop a new drug that can effectively treat arthritis is an important issue that the present invention intends to solve here.

In order to solve the above-mentioned problems, those skilled in the art urgently need to develop a novel medicament for treating arthritis for the benefit of a large group of people in need thereof.

### SUMMARY OF THE INVENTION

A primary objective of the present invention is to provide a method for preparing a composition comprising a mesenchymal stem cell (MSC), an extracellular vesicle (EV) produced by the mesenchymal stem cell, and a growth factor, comprising the following steps: (a) providing the mesenchymal stem cell; (b) culturing the mesenchymal stem cell in a keratinocyte serum-free medium (SFM) supplemented with N-acetyl-L-cysteine and L-ascorbic acid 2-phosphate, thereby expanding the mesenchymal stem cell; and (c) standing the mesenchymal stem cell for a predetermined time to obtain the composition comprising the mesenchymal stem cell, the extracellular vesicle produced by the mesenchymal stem cell, and the growth factor.

According to an embodiment of the present invention, the mesenchymal stem cell is human adipose-derived mesenchymal stem cell (ADSC).

According to an embodiment of the present invention, the N-acetyl-L-cysteine has a concentration of 1-100 mM, and the L-ascorbic acid 2-phosphate has a concentration of 0.05-50 mM.

According to an embodiment of the present invention, the method further comprises performing cell expanding of the mesenchymal stem cell in a culture flask made of a material with an oxygen-containing functional group at ratio of 20-35%.

According to an embodiment of the present invention, the predetermined time is 18-24 hours.

According to an embodiment of the present invention, the growth factor is uteroglobin.

According to an embodiment of the present invention, the growth factor is selected from the group consisting of: granulocyte colony-stimulating factor (G-CSF), interleukin-10 (IL-10), platelet-derived growth factor-AA (PDGF-AA), transforming growth factor-α (TGFα), vascular endothelial growth factor (VEGF-A), cartilage oligomeric matrix protein (COMP), IL-18 binding protein-α (IL-18BPα), angiopoietin-like 3 (ANGPTL-3), matrix metalloproteinase-2 (MMP-2), and a combination thereof.

According to an embodiment of the present invention, the growth factor is IL-10 or TGFα, and expressions of the IL-10 and the TGFα are downregulated.

Another objective of the present invention is to provide a composition comprising a mesenchymal stem cell (MSC), an extracellular vesicle (EV) produced by the mesenchymal stem cell, and a growth factor, which is prepared by the aforementioned method.

Another objective of the present invention is to provide a method for treating arthritis, comprising administering to a subject in need thereof a medicament comprising an effective amount of the aforementioned composition.

According to an embodiment of the present invention, the arthritis is degenerative arthritis.

According to an embodiment of the present invention, the mesenchymal stem cell has an effective concentration of single intra-articular injection of 6×10⁶-5×10⁷ mesenchymal stem cells.

According to an embodiment of the present invention, the composition increases cartilage thickness.

According to an embodiment of the present invention, the composition improves arthritis pain, stiffness and body function.

According to an embodiment of the present invention, the human ADSC is expanded by using the keratinocyte serum-free medium (SFM) supplemented with 1-100 mM N-acetyl-L-cysteine and 0.05-50 mM L-ascorbic acid 2-phosphate.

According to an embodiment of the present invention, the human ADSC is further expanded in a culture flask made of a material with an oxygen-containing functional group at ratio of 20-35%.

According to an embodiment of the present invention, the arthritis is cured one week after the medicament is administered.

According to an embodiment of the present invention, the medicament is in a dosage form for parenteral administration.

In summary, the composition comprising the mesenchymal stem cell (MSC), the extracellular vesicle (EV) produced by the mesenchymal stem cell, and the growth factor of the present invention achieves the effect on increasing cartilage thickness, improving arthritis pain, stiffness and body function. Through a specific method of culturing stem cells, the cell production is increased, and the therapeutic effect is more than 40% one week ahead of treatment, while improving the quality of life of patients with osteoarthritis.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

The FIGURE shows the changes in cartilage thickness before and after treatment of the present invention evaluated by magnetic resonance imaging (MRI).

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

Unless otherwise stated in the context, “a”, “the” and similar terms used in the specification (especially in the following claims) should be understood as including singular and plural forms.

According to the present invention, the term “adipose-derived stem cells (ADSCs)” refers to mesemchymal stem cells separated from fat, which are multipotent stem cells having high plasticity. After induction, they can be differentiated into cells of many different tissues.

According to the present invention, the extracellular vesicle comprises exosome and microvesicles.

As used herein, the term “treating” or “treatment” refers to alleviating, reducing, ameliorating, relieving, or controlling one or more clinical signs of a disease or disorder, and lowering, stopping, or reversing the progression of severity regarding the condition or symptom being treated.

According to the present invention, the medicament can be manufactured to a dosage form suitable for parenteral administration, using techniques well known to those skilled in the art, including, but not limited to, injection (e.g., sterile aqueous solution or dispersion), sterile powder, tablet, troche, lozenge, pill, capsule, dispersible powder or granule, solution, suspension, emulsion, syrup, elixir, slurry, and the like.

The medicament according to the present invention may be administered by a parenteral route selected from the group consisting of: intraperitoneal injection, subcutaneous injection, intraepidermal injection, intradermal injection, intramuscular injection, intravenous injection, and intralesional injection.

The medicament according to the present invention can comprise a pharmaceutically acceptable carrier which is widely used in pharmaceutical manufacturing technology. For example, the pharmaceutically acceptable carrier can comprise one or more reagents selected from the group consisting of solvent, emulsifier, suspending agent, decomposer, binding agent, excipient, stabilizing agent, chelating agent, diluent, gelling agent, preservative, lubricant, absorption delaying agent, liposome, and the like. The selection and quantity of these reagents fall within the scope of the professional literacy and routine techniques of those skilled in the art.

According to the present invention, the pharmaceutically acceptable carrier comprises a solvent selected from the group consisting of water, normal saline, phosphate buffered saline (PBS), sugar solution, aqueous solution containing alcohol, and combinations thereof.

### Examples 1-4

According to the present invention, the cells used in the following examples are human adipose-derived mesenchymal stem cells (ADSCs).

According to the present invention, liposuction was performed from healthy donors during abdominal surgery. 2-5 g of adipose tissue was collected from the subcutaneous fat of the abdominal wall. The liposuction operation takes about 1 hour and the wound is less than 1 cm. All donors provide written consent. Human adipose tissue was placed in Ca²⁺/Mg²⁺-free phosphate buffered saline (PBS) and immediately transferred to the laboratory.

According to the present invention, human adipose tissue was removed from shipping medium and placed in a Petri dish. The adipose tissue was washed 3 to 4 times using Ca²⁺/Mg²⁺-free phosphate buffered saline (PBS) and cut into small pieces (volume approximately 1-3 mm³). The tissue was dissociated with 0.1-0.3% collagenase in a temperature environment of 36.5-38.5° C. for 60 minutes. After collagenase digestion, centrifugation was performed at 20-25° C. and 500 g for 5-15 minutes to separate cells and undigested tissue fragments from stromal vascular fraction (SVF) pellets. The dissociated cells were collected and cultured at 36.5-38.5° C. in an incubator providing 5% CO₂. After 1 to 2 days of culture, the supernatant and debris were removed from the culture to obtain primary adipose-derived mesenchymal stem cells.

The primary adipose-derived mesenchymal stem cells were then expanded and cultured in different culture media. The culture media used in each example are as follows.

Example 1: 0.5×10⁵ adipose-derived mesenchymal stem cells were placed in a 6-well cell culture plate (Corning) comprising 1-100 mM N-acetyl-L-cysteine (Sigma), 0.05-50 mM L-ascorbic acid 2-phosphate (Sigma) in keratinocyte-serum free medium (SFM) (Gibco) for cell culture for 1, 4, and 7 days. The culture environment is a cell culture incubator with a temperature controlled at 36.5-38.5° C. and containing 5% carbon dioxide.

Example 2: 0.5×10⁵ adipose-derived mesenchymal stem cells were placed in a 6-well cell culture plate (Corning) comprising 1-10 mg/ml human serum albumin (Bio-Pure), 0.05-50 mM L-ascorbic acid 2-phosphate, and 1-40 mM sodium bicarbonate (Sigma) in DMEM/F12 medium (Gibco) for cell culture for 1, 4, and 7 days. The culture environment is a cell culture incubator with a temperature controlled at 36.5-38.5° C. and containing 5% carbon dioxide.

Example 3: 0.5×10⁵ adipose-derived mesenchymal stem cells were placed in a 6-well cell culture plate (Corning) comprising 1-10 mg/ml human serum albumin (Bio-Pure), 0.05-50 mM L-ascorbic acid 2-phosphate, 1-40 mM sodium bicarbonate (Sigma), and 5-15 mM HEPES (Sigma) in DMEM/F12 medium (Gibco) for cell culture for 1, 4, and 7 days. The culture environment is a cell culture incubator with a temperature controlled at 36.5-38.5° C. and containing 5% carbon dioxide.

Example 4: 0.5×10⁵ adipose-derived mesenchymal stem cells were placed in a 6-well cell culture plate (Corning) comprising 5-20 wt % fetal bovine serum (Hyclone) in DMEM/F12 medium (Gibco) for cell culture for 1, 4, and 7 days. The culture environment is a cell culture incubator with a temperature controlled at 36.5-38.5° C. and containing 5% carbon dioxide.

Number of cells was evaluated with ADAM-MC™ Automatic Cell Counter (Digital Bio, NanoEnTek Inc.). When the culture days were 1, 4, and 7 days, the number of cells in each example was analyzed, and the results were recorded in Table 1.

TABLE 1

| | | Example 1 | Example 2 | Example 3 | Example 4 |
|---|---|---|---|---|---|
| Num- | Day 1 | 0.457 × 10⁵ | 0.369 × 10⁵ | 0.432 × 10⁵ | 0.336 × 10⁵ |
| ber of | Day 4 | 5.534 × 10⁵ | 3.460 × 10⁵ | 4.174 × 10⁵ | 1.548 × 10⁵ |
| cells | Day 7 | 8.765 × 10⁵ | 6.808 × 10⁵ | 7.904 × 10⁵ | 1.860 × 10⁵ |

As shown in the results in Table 1, whether it is the 4th day or the 7th day, the number of cells in Example 1 is the highest, followed by Example 3, Example 2, and Example 4 in sequence. The number of cells in Example 4 is significantly less than that of the other three groups.

In addition, cluster of differentiation (CD) refers to cell surface markers that cells of different lineages express or disappear during different stages of normal differentiation and maturation and activation processes. The CD marker is a protein complex or glycoprotein on the cell membrane. CD markers have many uses and are often used as important receptors or ligands of cells. At the same time, they can be used as a surface marker for cell identification and isolation, and are widely involved in various stages of cells, including cell growth, cell differentiation, cell migration, etc. CD29 (integrin β1) is a multi-functional protein involved in cell-matrix adhesion, cell signaling, cellular defense, cell adhesion, protein binding, protein heterodimerization, receptor-mediated activity, wound repair, etc. In addition, CD29 plays an important role in maintaining the chondrocyte phenotype, preventing chondrocyte apoptosis, and regulating chondrocyte-specific gene expression. CD44 is the main receptor for hyaluronic acid, one of the main extracellular matrix (ECM) components of cartilage. CD44 plays a key role in maintaining cartilage homeostasis. CD44 regulates the expression of cartilage-related genes, thereby promoting the production of important markers of chondrocytes, such as type II collagen and aggrecan. CD73 (5′ecto-nucleotidase) is a regulator of immune function. CD90 (Thy-1) plays a key role in adipose-derived mesenchymal stem cell proliferation and metabolism by activating AKT and cyclin D1. CD105 (endoglin) or TGF-β receptor III can be relatively specific markers of adipose-derived mesenchymal stem cells. Studies have shown that adipose-derived mesenchymal stem cells (CD105⁺ADSCs) expressing CD105 have a higher growth rate and differentiation ability. CD105 on mesenchymal stem cells is involved in the regulation of TGF-β/Smad2 signaling pathway and chondrogenic differentiation. Therefore, on the 7th day of culture, the surface antigen expression levels of the adipose-derived mesenchymal stem cells obtained in Examples 1 to 3 were analyzed respectively. 100 μL of 1×10⁶ cells/mL adipose-derived mesenchymal stem cells were taken into a microcentrifuge tube, and fluorescently labeled CD73, CD90, CD105, CD14, CD19, CD34, CD45, HLA-DR (Becton Dickinson) antibodies were added at a ratio of 1:100 and mixed evenly. After standing in the dark, cell markers were analyzed using a BD AccuriC6 flow cytometer (Becton Dickinson). After the analysis was completed, the results were recorded in Table 2.

TABLE 2

| | | Example 1 | Example 2 | Example 3 |
|---|---|---|---|---|
| Surface | CD 73 | 99.96 | 99.81 | 99.66 |
| antigen | CD 90 | 100 | 99.98 | 99.94 |
| expression | CD 105 | 99.19 | 99.69 | 98.17 |
| level (%) | CD 14 | 0.06 | 0.07 | 0.15 |
| | CD 19 | 0.06 | 0.17 | 0.13 |
| | CD 34 | 0.07 | 0.29 | 0.23 |
| | CD 45 | 0.10 | 0.16 | 0.18 |
| | HLA-DR | 0.04 | 0.07 | 0.12 |

Further, surface antigens in each example were confirmed. High levels of specific mesenchymal stem cell markers CD73, CD90 and CD105 were expressed on the ADSCs cultured in Examples 1 to 3. The molecular expression levels of hematopoietic cell markers CD14, CD19, CD34, CD45 and HLA-DR were very low, consistent with the characteristics of adipose-derived mesenchymal stem cells. At the same time, CD105, a marker related to chondrogenic differentiation, has high levels of expression.

In addition, the doubling-time of the cells after the seventh day of culture in Examples 1 to 4 was compared, and the results obtained are as shown in Table 3.

TABLE 3

| | Example 1 | Example 2 | Example 3 | Example 4 |
|---|---|---|---|---|
| doubling-time (hour) | 20.01 | 22.30 | 22.00 | 32.67 |
| doubling-time | 38.8% | 31.7% | 32.7% | — |
| (Take Example 4 as | shorter | shorter | shorter | |
| 100%) | | | | |

The doubling time of Example 1 is 20.01 hours, and the doubling times of Examples 2 to 4 are 22.30 hours, 22.00 hours and 32.67 hours respectively.

From the above results, it can be seen that the culture medium used in Example 1 can more effectively increase the expansion number of adipose-derived mesenchymal stem cells than the culture medium used in other examples, and would not affect cell activity and surface antigen characteristics. Also, high-level expression of CD105, a marker related to chondrogenic differentiation, can be seen.

### Examples 5 and 6

In this embodiment, the primary adipose-derived mesenchymal stem cells obtained in the same manner as in the aforementioned Examples 1 to 4 were placed in a culture flask made of a material with an oxygen-containing functional group at ratio of 20-35% and an oxygen-containing functional group at ratio of 5-20% material culture flaskes for cell expansion. The outer material of the culture flask whose oxygen-containing functional group ratio is 20-35% is polystyrene. Due to the incorporation of oxygen-containing functional groups on the polystyrene surface, the culture surface has a net negative surface charge, making the surface of the culture flask more hydrophilic and moist, which helps cell attachment and growth. The culture methods of each embodiment are as follows.

Example 5: 1×10⁶ adipose-derived mesenchymal stem cells were cultured in a culture flask (HYPERFlask, Corning) with an oxygen-containing functional group at ratio of 29% using the same culture medium components as Example 1 for 10 to 14 days. The number of cells is about 7×10⁷-3.146×10⁸. The culture environment is a cell culture incubator with a temperature controlled at 36.5-38.5° C. and containing 5% carbon dioxide.

Example 6: 1×10⁶ adipose-derived mesenchymal stem cells were cultured in a culture flask (175T Flask, Corning) with an oxygen-containing functional group at ratio of 17.2% using the same culture medium components as Example 1 for 10 to 14 days. The culture environment is a cell culture incubator with a temperature controlled at 36.5-38.5° C. and containing 5% carbon dioxide.

When culturing for 10 to 14 days, the cell number, cell survival rate, and surface antigen expression level of Examples 5 and 6 were analyzed respectively, and the results were recorded in Table 4.

TABLE 4

| | | Example 5 | Example 6 |
|---|---|---|---|
| The number of cells obtained per cm² | | 49,612 | 31,761 |
| cell survival rate | | 95% | 96% |
| surface antigen | CD 29 | 99.99 | 99.95 |
| expression level (%) | CD44 | 99.96 | 99.96 |
| | CD 90 | 100 | 99.98 |
| | CD 105 | 97.82 | 97.13 |
| | CD 14 | 0.00 | 0.02 |
| | CD 34 | 0.03 | 0.01 |
| | CD 45 | 0.02 | 0.03 |
| | HLA-DR | 0.13 | 0.15 |

From the results in Table 4 above, it can be seen that the number of cells in Example 5 increased from 1×10⁶ cells to 2.56×10⁸ cells (49,612 cells/cm²). The number of cells in Example 6 ranged from 1×10⁶ cells to 1.64×10⁸ cells (31,761 cells/cm²). It can be seen that under the same culture medium components, when adipose-derived mesenchymal stem cells were cultured in a culture flask made of a material with an oxygen-containing functional groups at ratio of 20-35%, the expansion speed would be significantly faster than that of a culture flask cultured with an oxygen-containing functional group at ratio of 5-20%.

Further, the surface antigens of each example were confirmed. High levels of specific mesenchymal stem cell markers CD90 and CD105 were expressed on the ADSCs cultured in Examples 5 and 6. The expression levels of hematopoietic cell markers CD14, CD34, CD45 and HLA-DR molecules were very low, consistent with the characteristics of adipose-derived mesenchymal stem cells.

From the above results, it can be seen that the culture flask used in Example 5 can more effectively increase the expansion number of adipose-derived mesenchymal stem cells than the culture flask used in Example 6, and would not affect cell activity and surface antigen characteristics. At the same time, markers CD29, CD44 and CD105 related to chondrogenesis and differentiation were expressed at high levels.

### Example 7 and Comparative Example 1

In this embodiment, the primary adipose-derived mesenchymal stem cells obtained in the same manner as in the aforementioned Examples 1 and 3 are used for cell expansion. The culture method is as follows.

Example 7: 1×10⁷ adipose-derived mesenchymal stem cells were cultured in a culture flask with an oxygen-containing functional group at ratio of 20-35% using the same culture medium components as Example 1 for 7 days. The culture environment is a cell culture incubator with a temperature controlled at 36.5-38.5° C. and containing 5% carbon dioxide.

Comparative Example 1: 1×10⁷ adipose-derived mesenchymal stem cells were cultured in a culture flask with an oxygen-containing functional group at ratio of 20-35% using the same culture medium components as Example 3 for 7 days. The culture environment is a cell culture incubator with a temperature controlled at 36.5-38.5° C. and containing 5% carbon dioxide.

When cultured to the 7th day, the cell numbers of each embodiment were analyzed respectively. As a result, it was found that the number of cells in Example 7 increased from 1×10⁷ cells to 7.88×10⁷ cells, while the number of cells in Comparative Example 1 decreased from 1×10⁷ cells to 1.96×10⁶ cells. It can be seen from this result that not all culture media and culture flaskes made of materials with an oxygen-containing functional group at ratio of more than 20% can expand the number of adipose-derived mesenchymal stem cells. The number of adipose-derived mesenchymal stem cells can be most efficiently expanded by using the culture medium of Example 1 and a culture flask made of a material with an oxygen-containing functional group at ratio of 20-35% (Example 7).

### Stability Test

The adipose-derived mesenchymal stem cells expanded in Example 7 were put into a cell cryopreservation solution [5-15% dimethyl sulfoxide (DMSO) or DMSO-containing cryopreservation solution] for aliquots, followed by placing the cryovial in an environment with a temperature of −196±10° C. and freezing it for storage. The cryovial containing the frozen cells was taken out according to the different freezing days in Table 5, and immediately placed in an incubator with a temperature of 37° C. for 3 minutes. The cryovial was transferred to a biological safety cabinet (BSC), the cryovial was opened, the cell suspension was transferred to a centrifuge tube, and centrifugation was performed at 300×g for 5 minutes.

The supernatant was removed and the cell pellet was gently placed in 10 mL of saline, followed by centrifuging for 5 minutes at 20-25° C. and removing the supernatant. After repeating this step three times, the cell survival rate was evaluated with an ADAM-MC™ Automatic Cell Counter (Digital Bio, NanoEnTek Inc.), and the results were recorded in Table 5.

TABLE 5

| Freezing days | Cell survival rate |
|---|---|
| 125 | 90% |
| 188 | 93% |
| 215 | 93% |
| 243 | 94% |
| 250 | 91% |
| 397 | 92% |
| 404 | 94% |
| 425 | 92% |
| 586 | 92% |
| 607 | 94% |
| 614 | 94% |
| 803 | 92% |
| 817 | 92% |
| 831 | 91% |
| 838 | 91% |
| 887 | 93% |
| 915 | 93% |
| 922 | 91% |
| 957 | 91% |
| 985 | 91% |
| 1,084 | 93% |

As shown in Table 5, after the cells cultured in a culture flask made of a material with an oxygen-containing functional group at ratio of 20-35% (Example 7) were cultured in the medium of Example 1 and cryopreserved for 1,084 days, the cell survival rate was maintained at over 90%. In summary, the experimental results show that the present invention is the most efficient way to expand the number of adipose-derived mesenchymal stem cells without affecting cell activity and surface antigen characteristics. At the same time, it can maintain more than 90% cell survival rate and high-level expression of markers related to chondrogenesis and differentiation for a long time, providing broad benefits for the future application and development of the product.

After thawing the cryovial of adipose-derived mesenchymal stem cells expanded in Example 7, 4-5×10⁷ cells were taken and mixed with 3 mL of phosphate buffered saline (DPBS) or saline. After standing for 0 to 48 hours in an environment of 2-10° C., centrifugation was performed at 300×g for 5 minutes to collect the supernatant and the extracellular vesicles (EVs) in the supernatant. The supernatant containing EVs was reacted with Exo-Quick-TC (System Biosciences, Palo Alto, CA, USA) at 4° C. for at least 12 hours, and then EVs were precipitated. After completing the precipitation of EVs, treated with protein lysis buffer to obtain protein of EVs. The protein concentration of EVs was 478 μg/mL after 18 to 24 hours and 404.5 g/mL after 48 hours. The supernatant and protein of EVs were analyzed for granulocyte colony-stimulating factor (G-CSF), interleukin-2 (IL-2), interleukin-10 (IL-10), platelet-derived growth factor-AA (PDGF-AA), transforming growth factor-α (TGFα), vascular endothelial growth factor (VEGF-A), cartilage oligomeric matrix protein (COMP), uteroglobin, IL-18binding protein-α (IL-18BPα), a-fetoprotein (AFP), angiopoietin-like 3 (ANGPTL-3), ANGPTL6/AGF, fatty acid-binding protein-1 (FABP1), fibroblast growth factor-19 (FGF-19), FGF-21, FGF-23, and matrix metalloproteinase-2 (MMP-2) content using MILLIPLEX® MAP MULIPLEX DETECTION (Merck Milliplex, instrument model: Luminex Magpix analyzer). The values are recorded in Table 6.

TABLE 6

| | Example 7 | | | | | |
|---|---|---|---|---|---|---|
| | 0 hr | | 24 hrs | | 48 hrs | |
| Factor (pg/ml) | Supernatant | EVs | Supernatant | EVs | Supernatant | EVs |
| G-CSF | 2.54 ± 0.53 | 3.34 ± 0.81 | 150.48 ± 0.00 | 123.84 ± 1.10 | 116.31 ± 3.10 | 67.38 ± 4.79 |
| IL-2 | 0.41 ± 0.03 | 0.38 ± 0.03 | 0.28 ± 0.00 | 0.39 ± 0.00 | 0.32 ± 0.00 | 0.45 ± 0.03 |
| IL-10 | 1.83 ± 0.13 | 1.77 ± 0.09 | 1.57 ± 0.00 | 1.57 ± 0.00 | 1.08 ± 0.00 | 1.24 ± 0.00 |
| PDGF-AA | 7.19 ± 0.20 | 8.89 ± 1.53 | 16.19 ± 0.74 | 10.52 ± 0.23 | 10.53 ± 0.67 | 24.47 ± 0.00 |
| TGFα | 0.93 ± 0.07 | 1.04 ± 0.11 | 0.64 ± 0.00 | 0.95 ± 0.21 | 0.64 ± 0.00 | 0.71 ± 0.00 |
| VEGF-A | 1.60 ± 0.08 | 1.50 ± 0.08 | 34.59 ± 1.85 | 270.05 ± 2.40 | 27.91 ± 1.55 | 118.52 ± 2.11 |
| COMP | 3.64 ± 0.00 | 4.00 ± 0.32 | 4.51 ± 0.00 | 3.64 ± 0.00 | 4.51 ± 0.00 | 5.19 ± 0.33 |
| Uteroglobin | 2.42 ± 0.00 | 2.56 ± 0.06 | 2.77 ± 0.00 | 2.60 ± 0.25 | 2.69 ± 0.12 | 3.13 ± 0.00 |
| IL-18BPα | 1.42 ± 0.00 | 0.99 ± 0.38 | 2.06 ± 0.00 | 1.42 ± 0.00 | 1.74 ± 1.45 | 1.74 ± 1.45 |
| AFP | 0.01 ± 0.00 | 0.01 ± 0.00 | 0.01 ± 0.00 | 0.01 ± 0.00 | 0.01 ± 0.00 | 0.01 ± 0.00 |
| ANGPTL3 | 0.34 ± 0.01 | 0.34 ± 0.01 | 0.26 ± 0.01 | 0.38 ± 0.01 | 0.30 ± 0.01 | 0.38 ± 0.01 |
| ANGPTL6/AGF | 0.48 ± 0.01 | 0.54 ± 0.05 | 0.48 ± 0.02 | 0.48 ± 0.02 | 0.43 ± 0.02 | 0.50 ± 0.01 |
| FABP1 | 0.02 ± 0.00 | 0.02 ± 0.00 | 0.02 ± 0.00 | 0.02 ± 0.00 | 0.02 ± 0.00 | 0.02 ± 0.00 |
| FGF-19 | 0.05 ± 0.00 | 0.07 ± 0.02 | 0.06 ± 0.00 | 0.06 ± 0.00 | 0.06 ± 0.00 | 0.09 ± 0.00 |
| FGF-21 | 0.00 ± 0.00 | 0.00 ± 0.00 | 0.00 ± 0.00 | 0.00 ± 0.00 | 0.00 ± 0.00 | 0.00 ± 0.00 |
| FGF-23 | 0.01 ± 0.00 | 0.01 ± 0.00 | 0.01 ± 0.00 | 0.01 ± 0.00 | 0.01 ± 0.00 | 0.01 ± 0.00 |
| MMP-2 | 83.56 ± 0.00 | 98.75 ± 13.59 | 732.88 ± 0.00 | 512.95 ± 62.11 | 669.83 ± 89.17 | 310.66 ± 0.00 |

### Composition of Present Invention

The adipose-derived mesenchymal stem cells expanded in Example 7 were mixed with 3.3±0.2 mL of phosphate buffered saline (DPBS) or saline (saline), followed by standing for 18 to 24 hours in an environment of 2-10° C. During the standing process, the adipose-derived mesenchymal stem cells expanded in Example 7 would release extracellular vesicles and growth factors. As mentioned above, the preparation composed of adipose-derived mesenchymal stem cells, extracellular vesicles and growth factors produced during a specific resting time is the composition of the present invention, which can be used to treat degenerative arthritis.

In addition, adipose-derived mesenchymal stem cells can also be placed in other injection water until they produce extracellular vesicles and growth factors. For example, it can be selected from distilled water for injection, 0.45-3% sodium chloride injection, 2.5-50% glucose injection, Lactated Ringer's B injection, or Ringer's Solution, there is no limitation here.

### Application Example

### Composition of Present Invention Treats Degenerative Arthritis

In this application example, the composition of the present invention obtained above is used for the clinical treatment of patients with degenerative arthritis. This application example is conducted in accordance with the ethical principles of the Declaration of Helsinki and local laws and regulations. This application example follows the current Good Clinical Trial Practice Guidelines for Drugs.

The quality testing related to stem cells in this application example is performed by a third-party certification laboratory [Taiwan Accreditation Foundation (TAF), certification standard: ISO/IEC 17025, certification number: 2800]. The sterility test is based on the Chinese Pharmacopoeia sterility test method and USP43, Sterility Tests, and is evaluated by the direct inoculation method. Gram stain is another rapid microbial detection test that uses staining to distinguish Gram positive/negative bacteria. Mycoplasma is evaluated using nucleic acid amplification technology based on the Chinese Pharmacopoeia Mycoplasma test method. Endotoxin testing is based on the Chinese Pharmacopoeia Bacterial Endotoxins Testing Method and USP43, Bacterial Endotoxins Tests, and is evaluated by kinetic colorimetry. Cell surface markers CD34, CD45, CD90, and CD105 (Becton Dickinson) were analyzed using a BD AccuriC6 flow cytometer (Becton Dickinson). Cell survival rate was assessed with ADAM-MC™ Automatic Cell Counter (Digital Bio, NanoEnTek Inc.).

The standards after adipose-derived mesenchymal stem cell expansion are shown in Table 7.

TABLE 7

| Indications | | Osteoarthritis |
|---|---|---|
| Stem cell source * | | Fat |
| Number of cells | | 6-7 × 10⁶ ADSCs |
| | | 4-5 × 10⁷ ADSCs |
| Cell activity (%) | | >70 |
| Cell surface antigen | CD34 | <10 |
| expression (%) | CD45 | <10 |
| | CD90 | >90 |
| | CD105 | >90 |

Safety

| | |
|---|---|
| Microbiological testing | not detectable |
| Endotoxin testing (EU/mL) | <0.25 |
| Mycoplasma test | non-reactive |
| Excipients | 3.3 ± 0.2 mL of saline |
| Storage and resting conditions | 2-10° C., 0-24 hours |

* The specific requirements for adipose tissue donors are that they are between the ages of 20 and 70 and do not have the risk of relevant communicable disease agents or diseases (RCSADs), and there is also no risk of disease transmission from allogeneic transplants; Test results for HIV type 1 & 2, hepatitis B virus (HBV), hepatitis C virus (HCV), Treponema pallidum, human transmissible spongiform encephalopathy (TSE) and other related infectious pathogens or diseases are all negative or non-reactive.

The selection conditions for subjects are as follows.

Inclusion Conditions:
1. Signed and dated subject consent form are provided.
2. The age of the subjects is from 40 to 80 years old (including 80 years old).
3. The subject's knee joint degeneration grade (Kellgren-Lawrence Grading Scale) is from grade 2 to grade 4, which is evaluated using the knee arthritis criteria set by the American College of Rheumatology.
4. Even while taking nonsteroidal anti-inflammatory drug (NSAID) therapy, the subject's target knee still had a Western Ontario and McMaster Universities Osteoarthritis Index (WOMAC) between 7 and 17 (inclusive).
5. Those who cannot tolerate long-term treatment with nonsteroidal anti-inflammatory drugs (NSAIDs). For example, you have experienced serious gastrointestinal side effects due to the use of NSAIDs or have an underlying disease that increases the risk to the gastrointestinal system, cardiovascular system or kidneys after using NSAIDs. Those who cannot take or wish to delay knee replacement surgery.

Exclusion Criteria:
1. The target knee has undergone surgery for fractures, ligament reconstruction, semilunar cartilage reconstruction, and knee replacement surgery.
2. Intra-articular interventional treatment (e.g., steroids, anesthetics, hyaluronic acid, and arthroscopic surgery) of the target knee in the 12 weeks prior to screening.
3. Within 1 week before screening, except acetaminophen and non-steroidal anti-inflammatory drugs (NSAIDs), subject receives or requires treatment with systemic or local immunosuppressants, anti-inflammatory drugs, steroids, analgesics, opioid analgesics, or duloxetine (antidepressant) in the target knee joint.
4. Suffering from other joint diseases other than knee osteoarthritis, the trial moderator determines that you are not eligible to participate in the trial.
5. Unable to undergo magnetic resonance imaging (MRI) examination, including allergy to the magnetic resonance imaging (MRI) imaging agent used in the trial, known to have claustrophobia, the presence of any metallic intraocular foreign body or active/inactive implantable medical devices (e.g., cardiac pacemaker, cochlear (artificial electronic ear), intracranial vascular clamp or neurostimulator).
6. The target knee joint is infected or suspected to be infected.
7. Have a history of human immunodeficiency virus (HIV) infection.
8. History of malignant tumors within 2 years before screening.
9. Body mass index (BMI)≥35 kg/m² (kg/height in meters squared).
10. Known allergy to any ingredient of this test product or active control.
11. Participated in other pilot trials within 4 weeks before screening.
12. Serious medical condition that persists or has occurred within the past 2 years (e.g. co-existing medical conditions): cardiovascular disease (such as New York Heart Association class III or IV), liver disease (e.g., Child-Pugh Class C), mental illness (e.g., alcoholism and drug abuse), medical history, physical examination, or laboratory test abnormalities, the trial moderator determines that it may interfere with the trial results or adversely affect the safety of the subjects.
13. Women of childbearing potential who are breastfeeding or have a positive serum/urine pregnancy test result at the time of screening.
14. A subject of childbearing potential refuses to use highly effective contraception from the time of signing the subject consent form to the final/early termination visit. At least two methods of contraception must be used, one of which must be a barrier method. Acceptable ways include:
(1) Regular and continued use of oral, injectable, or implantable hormonal contraceptive methods.
(2) Insertion of an intrauterine device (IUD) or intrauterine system (IUS).
(3) Barrier methods: Condoms or blocking caps (diaphragm or cervical/top cap).

Each subject is screened according to the above inclusion and exclusion conditions. Eligible subjects received the composition comprising 6-7×10⁶ or 4-5×10⁷ adipose-derived mesenchymal stem cells, extracellular vesicles generated from the adipose-derived mesenchymal stem cells, and growth factors with single intra-articular injection on the first day at the target knee, low dose and high dose, respectively. Visits are scheduled at weeks 1, 4, 8, 12, 24, 36 and 48. Subjects were asked to perform the Western Ontario and McMaster Universities Osteoarthritis Index (WOMAC), Visual Analog Scales (VAS), and quality of life scale SF-12 health questionnaire, and changes in regular blood, vital signs, and target knee MRI assessments were tracked after injection. The observations are shown in Table 8.

The basic information of the subjects, including age, weight, height, BMI, gender, race, illness time and Kellgren-Lawrence grading method (KL Grade) results are recorded in Table 9. All subjects did not experience any serious adverse events (SAE) related to the trial drug.

TABLE 9

Treatment

| Characteristics | Low dose | High dose |
|---|---|---|
| Age [Y/O] | | |
| Min-Max | 57.0-73.0 | 52.0-72.0 |
| Body Weight [kg] | | |
| Min-Max | 53.6-84.0 | 57.6-87.0 |
| Body Height [cm] | | |
| Min-Max | 152.8-166.2 | 147.3-172.9 |
| Baseline BMI [kg/m²] | | |
| Min-Max | 20.1-30.4 | 21.7-32.9 |
| Gender | | |
| Male | 0% | 40.0% |
| Female | 100.0% | 60.0% |
| Target Knee | | |
| Left Knee | 40.0% | 80.0% |
| Right Knee | 60.0% | 20.0% |
| Race | | |
| Asian | 100.0% | 100.0% |

Disease Duration [years]

| | | |
|---|---|---|
| Min-Max | 1.69-9.85 | 0.07-10.25 |

Kellgren-Lawrence Grade (KL Grade)on Target Knee

| | | |
|---|---|---|
| Grade 0 | 0% | 0% |
| Grade 1 | 0% | 0% |
| Grade 2 | 40.0% | 46.7% |
| Grade 3 | 40.0% | 53.3% |
| Grade 4 | 20.0% | 0% |

WOMAC baseline

| | | |
|---|---|---|
| Min-Max | 30-47 | 38-56 |

WOMAC pain baseline

| | | |
|---|---|---|
| Min-Max | 9-10 | 9-12 |

WOMAC stiffness baseline

| | | |
|---|---|---|
| Min-Max | 1-4 | 3-6 |

WOMAC physical function baseline

| | | |
|---|---|---|
| Min~Max | 20-34 | 29-42 |

VAS baseline

| | | |
|---|---|---|
| Min-Max | 19-74 | 52-83 |

SF-12 PCS baseline

| | | |
|---|---|---|
| Min-Max | 34.34-43.81 | 21.62-40.69 |

SF-12 MCS baseline

| | | |
|---|---|---|
| Min-Max | 39.87-48.05 | 36.13-50.79 |

SF-12 SF6D_R2 baseline

| | | |
|---|---|---|
| Min-Max | 0.48-0.8 | 0.52-0.818 |

Cartilage thickness (mm)

| | | |
|---|---|---|
| Min-Max | 1.10-1.87 | 1.13-2.23 |

WOMAC is a questionnaire proposed by Bellamy et al. in 1981 to assess the health status of hip joints and knee joints. It is divided into three parts: pain, stiffness, and joint function to evaluate the structure and function of the hip and knee joints. There are a total of 24 items. Each item ranges from the mildest (0 points) to the most severe (4 points). There are 5 items for the pain part, 2 items for the stiffness part, and 17 items for the joint function part. The maximum total score is 96 points, with WOMAC pain ranging from 0 to 20 points, WOMAC stiffness ranging from 0 to 8 points, and WOMAC physiological function ranging from 0 to 68 points. Research shows that this scale has objective reliability, validity and sensitivity for the assessment of degenerative knee arthritis, and is an assessment scale widely used in patients with degenerative arthritis. The present invention uses a visual analog scale to assess patients' pain levels. This method is widely used clinically to track patients' musculoskeletal system pain. The test method is to prepare a blank paper with a 100 cm long straight line on it, with the left endpoint marked 0 and the right endpoint marked 100. During the test, patients would be told: 0 means no pain at all and 100 means the most unbearable pain. The patient is asked to take a pen and mark the degree of pain on a straight line. The pain index can be expressed by measuring the length with a graduated ruler (in mm). As shown in Tables 10 to 13, after 1 week of treatment, patients in both the low-dose group and the high-dose group significantly reduced pain by at least 35%. At high doses, the pain relief can last for at least 24 weeks by more than 80%. Stiffness caused by osteoarthritis, after 1 week of treatment, was significantly improved by at least 25% in both the low-dose group and the high-dose group, and continued to be improved by more than 55% for at least 24 weeks. In the part of body function abnormalities caused by osteoarthritis, after 1 week of treatment, both the low-dose group and the high-dose group were significantly improved by at least 43%, and continued to be at least 52% for at least 24 weeks. In summary, it can be learned that the present invention can effectively improve the pain, stiffness and physical function of osteoarthritis one week after treatment, and last for at least 24 weeks.

TABLE 10
The WOMAC and VAS results of the low-dose group at 0, 1, 4, 8, 12, and 24 weeks after treatment

| | 0 wk | | 1 wk | | 4 wk | | 8 wk | | 12 wk | | 24 wk | |
|---|---|---|---|---|---|---|---|---|---|---|---|---|
| NET CHANGE | Mean | SD | Mean | SD | Mean | SD | Mean | SD | Mean | SD | Mean | SD |
| WOMAC | 0.00 | 0.00 | −17.67 | 2.08 | −4.33 | 4.73 | −16.33 | 5.51 | −17.33 | 8.50 | −18.00 | 18.38 |
| WOMAC pain | 0.00 | 0.00 | −4.67 | 0.58 | −2.33 | 1.53 | −4.33 | 1.15 | −5.00 | 1.73 | −5.50 | 3.54 |
| WOMAC | 0.00 | 0.00 | −0.67 | 0.58 | −0.33 | 0.58 | −1.00 | 1.00 | −0.67 | 0.58 | −1.50 | 0.71 |
| stifness | | | | | | | | | | | | |
| WOMAC | 0.00 | 0.00 | −12.00 | 2.00 | −2.33 | 4.51 | −10.67 | 3.79 | −11.67 | 6.66 | −14.00 | 18.38 |
| physical function | | | | | | | | | | | | |
| VAS | 0.00 | 0.00 | −28.00 | 17.06 | −21.00 | 11.53 | −22.33 | 4.51 | −13.67 | 8.08 | −14.00 | 4.24 |

TABLE 11
The improvement percentage of WOMAC and VAS in the low-dose
group at 0, 1, 4, 8, 12, and 24 weeks after treatment

| improved % | 0 wk | 1 wk | 4 wk | 8 wk | 12 wk | 24 wk |
|---|---|---|---|---|---|---|
| WOMAC | 0% | 45% | 11% | 42% | 44% | 46% |
| WOMAC pain | 0% | 50% | 25% | 46% | 54% | 59% |
| WOMAC stifness | 0% | 25% | 13% | 38% | 25% | 56% |
| WOMAC physical | 0% | 44% | 9% | 40% | 43% | 52% |
| function | | | | | | |
| VAS | 0% | 54% | 40% | 43% | 26% | 27% |

TABLE 12
WOMAC and VAS results of the high-dose group at 0, 1, 4, 8, 12, and 24 weeks after treatment

| NET | 0 wk | | 1 wk | | 4 wk | | 8 wk | | 12 wk | | 24 wk | |
|---|---|---|---|---|---|---|---|---|---|---|---|---|
| CHANGE | Mean | SD | Mean | SD | Mean | SD | Mean | SD | Mean | SD | Mean | SD |
| WOMAC | 0.00 | 0.00 | −19.83 | 8.75 | −28.33 | 8.26 | −30.33 | 6.06 | −33.00 | 8.49 | −36.80 | 8.32 |
| WOMAC pain | 0.00 | 0.00 | −4.13 | 1.46 | −5.50 | 0.93 | −6.13 | 1.55 | −6.38 | 1.69 | −7.57 | 1.62 |
| WOMAC | 0.00 | 0.00 | −1.89 | 1.17 | −2.00 | 0.87 | −2.44 | 0.88 | −2.44 | 0.73 | −2.38 | 0.92 |
| stifness | | | | | | | | | | | | |
| WOMAC | 0.00 | 0.00 | −15.25 | 4.40 | −19.25 | 6.14 | −20.38 | 4.47 | −20.25 | 7.78 | −23.75 | 6.63 |
| physical | | | | | | | | | | | | |
| function | | | | | | | | | | | | |
| VAS | 0.00 | 0.00 | −22.67 | 14.20 | −32.67 | 15.26 | −35.78 | 13.48 | −39.33 | 15.03 | −53.25 | 9.57 |

TABLE 13
The improvement percentage of WOMAC and VAS in the high-dose
group at 0, 1, 4, 8, 12, and 24 weeks after treatment

| improved % | 0 wk | 1 wk | 4 wk | 8 wk | 12 wk | 24 wk |
|---|---|---|---|---|---|---|
| WOMAC | 0% | 44% | 63% | 67% | 73% | 81% |
| WOMAC pain | 0% | 42% | 56% | 63% | 65% | 78% |
| WOMAC stifness | 0% | 44% | 46% | 56% | 56% | 55% |
| WOMAC physical | 0% | 43% | 55% | 58% | 58% | 68% |
| function | | | | | | |
| VAS | 0% | 35% | 51% | 56% | 61% | 83% |

SF-12 was developed by Ware et al. in 1994 and was used in more than one million studies within a year, and also selected for the Annual Member Health Care Survey by the National Committee 32 for Quality Assurance (NCQA). For patients with chronic pain, long-term pain may result in reduced quality of life and changes in personal physical and mental health. In order to understand whether subjects have experienced changes in pain levels after treatment, which in turn affects changes in quality of life, various quality of life scales are often used as assessment tools in quality-of-life assessment studies around the world.

The SF-12 questionnaire mainly measures the recipient's perceived health status, including two major factors of physical and mental health status, and comprises eight assessment concepts: general health (GH), physical function (PF), role physical (RP), bodily pain (BP), vitality (VT), social function (SF), role emotional (RE), and mental health (MH). In addition, the 12 questions can be divided into two major aspects: physical component summary (PCS) and mental component summary (MCS), and points would be assigned to each. This questionnaire is used to understand the subject's physical and mental health status in the past month. The higher the score, the better the subject's mental or physical health status. The scoring of this questionnaire needs to be converted and calculated with reference to the norms of the questionnaire to obtain the correct score. A derivative of SF-12 is SF-6D, which is a multi-attribute utility measure composed of 6 dimensions (physical functioning, role limitations, social functioning, pain, energy, and mental health). Each dimension has between four and six levels. The utility value of SF-6D health status is 1.0 for full health and 0 for death. As shown in Tables 14 to 17, after 1 week of treatment, patients in both the low-dose group and the high-dose group improved their quality of life by at least 7%. At the high dose, the improvement in quality of life can last for at least 24 weeks by more than 22%. In summary, it can be known that the present invention can effectively improve the quality of life one week after treatment and last for at least 24 weeks.

TABLE 14
SF-12 results of the low-dose group at 0, 1, 4, 8, 12, and 24 weeks after treatment

| | 0 wk | | 1 wk | | 4 wk | | 8 wk | | 12 wk | | 24 wk | |
|---|---|---|---|---|---|---|---|---|---|---|---|---|
| NET CHANGE | Mean | SD | Mean | SD | Mean | SD | Mean | SD | Mean | SD | Mean | SD |
| PCS | 0.00 | 0.00 | 2.99 | 4.62 | 3.35 | 4.40 | 2.89 | 2.88 | 7.12 | 3.11 | 10.32 | 9.05 |
| MCS | 0.00 | 0.00 | 6.24 | 6.00 | 1.07 | 5.85 | 4.85 | 1.32 | 5.54 | 2.04 | −2.59 | 4.17 |
| SF6D_R2 | 0.00 | 0.00 | 0.06 | 0.12 | 0.00 | 0.17 | 0.06 | 0.13 | 0.09 | 0.25 | 0.24 | 0.05 |

TABLE 15
SF-12 improvement percentage in the low-dose group
at 0, 1, 4, 8, 12, and 24 weeks after treatment

| improved % | 0 wk | 1 wk | 4 wk | 8 wk | 12 wk | 24 wk |
|---|---|---|---|---|---|---|
| PCS | 0% | 7% | 8% | 7% | 18% | 25% |
| MCS | 0% | 14% | 2% | 11% | 13% | −6% |
| SF6D_R2 | 0% | 9% | 0% | 10% | 14% | 37% |

TABLE 16
SF-12 results of the high-dose group at 0, 1, 4, 8, 12, and 24 weeks after treatment

| | 0 wk | | 1 wk | | 4 wk | | 8 wk | | 12 wk | | 24 wk | |
|---|---|---|---|---|---|---|---|---|---|---|---|---|
| NET CHANGE | Mean | SD | Mean | SD | Mean | SD | Mean | SD | Mean | SD | Mean | SD |
| PCS | 0.00 | 0.00 | 6.16 | 3.78 | 8.65 | 5.22 | 9.58 | 5.06 | 8.62 | 3.98 | 11.34 | 5.55 |
| MCS | 0.00 | 0.00 | 6.18 | 7.54 | 11.94 | 4.09 | 6.20 | 4.49 | 11.76 | 5.69 | 15.51 | 6.12 |
| SF6D_R2 | 0.00 | 0.00 | 0.05 | 0.07 | 0.02 | 0.23 | 0.07 | 0.04 | 0.08 | 0.08 | 0.14 | 0.08 |

TABLE 17
SF-12 improvement percentage in the high-dose group
at 0, 1, 4, 8, 12, and 24 weeks after treatment

| improved % | 0 wk | 1 wk | 4 wk | 8 wk | 12 wk | 24 wk |
|---|---|---|---|---|---|---|
| PCS | 0% | 18% | 26% | 29% | 26% | 34% |
| MCS | 0% | 15% | 28% | 15% | 28% | 37% |
| SF6D_R2 | 0% | 8% | 4% | 11% | 13% | 22% |

Magnetic resonance imaging (MRI) was used to evaluate the changes in cartilage thickness before and after treatment of the present invention. As shown in the FIGURE, after 24 weeks of treatment, the low-dose group increased cartilage thickness by 0.06 mm, an increase of 4% from the base period. The high-dose group increased cartilage thickness by 0.33 mm, an increase of 22% since the basal period. After 48 weeks of treatment, the low-dose group had an increase in cartilage thickness of 0.01 mm, an increase of 1% since the base period. The high-dose group increased cartilage thickness by 0.18 mm, an increase of 12% since the basal period. It can be seen that the present invention has the potential to increase cartilage thickness.

In summary, the composition comprising the mesenchymal stem cell (MSC), the extracellular vesicle (EV) produced by the mesenchymal stem cell, and the growth factor of the present invention achieves the effect on increasing cartilage thickness, improving arthritis pain, stiffness and body function. Through a specific method of culturing stem cells, the cell production is increased, and the therapeutic effect is more than 40% one week ahead of treatment, while improving the quality of life of patients with osteoarthritis.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for preparing a composition comprising a mesenchymal stem cell (MSC), an extracellular vesicle (EV) produced by the mesenchymal stem cell, and a growth factor, comprising the following steps:
(a) providing the mesenchymal stem cell;
(b) culturing the mesenchymal stem cell in a keratinocyte serum-free medium (SFM) supplemented with N-acetyl-L-cysteine and L-ascorbic acid 2-phosphate, thereby expanding the mesenchymal stem cell; and
(c) standing the mesenchymal stem cell for a predetermined time to obtain the composition comprising the mesenchymal stem cell, the extracellular vesicle produced by the mesenchymal stem cell, and the growth factor.

2. The method according to claim 1, wherein the mesenchymal stem cell is human adipose-derived mesenchymal stem cell (ADSC).

3. The method according to claim 1, wherein the N-acetyl-L-cysteine has a concentration of 1-100 mM, and the L-ascorbic acid 2-phosphate has a concentration of 0.05-50 mM.

4. The method according to claim 1, further comprising performing cell expanding of the mesenchymal stem cell in a culture flask made of a material with an oxygen-containing functional group at ratio of 20-35%.

5. The method according to claim 1, wherein the predetermined time is 18-24 hours.

6. The method according to claim 1, wherein the growth factor is uteroglobin.

7. The method according to claim 1, wherein the growth factor is selected from the group consisting of: granulocyte colony-stimulating factor (G-CSF), interleukin-10 (IL-10), platelet-derived growth factor-AA (PDGF-AA), transforming growth factor-α (TGFα), vascular endothelial growth factor (VEGF-A), cartilage oligomeric matrix protein (COMP), IL-18 binding protein-α (IL-18BPα), angiopoietin-like 3 (ANGPTL-3), matrix metalloproteinase-2 (MMP-2), and a combination thereof.

8. The method according to claim 7, wherein the growth factor is IL-10 or TGFα, and expressions of the IL-10 and the TGFα are downregulated.

9. A composition comprising a mesenchymal stem cell (MSC), an extracellular vesicle (EV) produced by the mesenchymal stem cell, and a growth factor, which is prepared by the method according to claim 1.

10. The composition according to claim 9, wherein the mesenchymal stem cell is human adipose-derived mesenchymal stem cell (ADSC).

11. A method for treating arthritis, comprising administering to a subject in need thereof a medicament comprising an effective amount of the composition according to claim 9.

12. The method according to claim 11, wherein the mesenchymal stem cell is human adipose-derived mesenchymal stem cell (ADSC).

13. The method according to claim 11, wherein the arthritis is degenerative arthritis.

14. The method according to claim 11, wherein the mesenchymal stem cell has an effective concentration of single intra-articular injection of 6×10⁶-5×10⁷ mesenchymal stem cells.

15. The method according to claim 11, wherein the composition increases cartilage thickness.

16. The method according to claim 11, wherein the composition improves arthritis pain, stiffness and body function.

17. The method according to claim 12, wherein the human ADSC is expanded by using the keratinocyte serum-free medium (SFM) supplemented with 1-100 mM N-acetyl-L-cysteine and 0.05-50 mM L-ascorbic acid 2-phosphate.

18. The method according to claim 17, wherein the human ADSC is further expanded in a culture flask made of a material with an oxygen-containing functional group at ratio of 20-35%.

19. The method according to claim 11, wherein the arthritis is cured one week after the medicament is administered.

20. The method according to claim 11, wherein the medicament is in a dosage form for parenteral administration.
